SEQUENCE LISTING (24 sequences)

SEQ ID NO: 1 - DNA, 2323 bp, Homosapien
ggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcaaaaaaaaaaaaaaaaaa

SEQ ID NO: 2 - protein, 592 aa, Homosapien
MWTLVSWVALTAGLVAGTRCPDGQFCPVACCLDPGGASYSCCRPLLDKPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLLQSKCLSKENATIDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 48 aa, Homosapien
CCGCGCTGGGACGCCCCTTIGAGGGACCCAGCCTIGAGACAGCTGCTG

SEQ ID NO: 4 - protein, 48 aa, Homosapien
CCGCGCTGGGACGCCCCTTTGAGGGACCCAGCCTTGAGACAGCTGCTG

SEQ ID NO: 5 - protein, 15 aa, Homosapien
HLSLPDPQALKRDVP

SEQ ID NO: 6 - protein, 45 aa, Homosapien
CACCTCAGCCTGCCAGACCCACAAGCCTIGAAGAGAGATGTCCCC

SEQ ID NO: 7 - protein, 20 aa, Homosapien
RREAPRWDAPLRDPALRQLL

SEQ ID NO: 8 - protein, 60 aa, Homosapien
CGCAGGGAGGCCCCGCGCTGGGACGCCCCTTTGAGGGACCCAGCCTTGAGACAGCTGCTG

SEQ ID NO: 9 - protein, 27 aa, Homosapien
QGPHQVPWMEKAPAHLSLPDPQALKRD

SEQ ID NO: 10 - protein, 81 aa, Homosapien
CAGGGGCCCCACCAGGTGCCCTGGATGGAGAAGGCCCCAGCTCACCTCAGCCTGCCAGACCCACAAGCCTTGAAGAGAGAT

SEQ ID NO: 11 - protein, 17 aa, Homosapien
MWTLVSWVALTAGLVAG

SEQ ID NO: 12 - protein, 51 aa, Homosapien
ATGTGGACCCTGGTGAGCTGGGTGGCCTTAACAGCAGGGCTGGTGGCTGGA

SEQ ID NO: 13 - protein, 33 aa, Homosapien
TRCPDGQFCPVACCLDPGGASYSCCRPLLDKWP

SEQ ID NO: 14 - protein, 120 aa, Homosapien
ACGCGGTGCCCAGATGGTCAGTTCTGCCCTGTGGCCTGCTGCCTGGACCCCGGAGGAGCCAGCTACAGCTGCTGCCGTCCCCTTCTGGACAAATGGCCCACAACACTGAGCAGGCATCTG

SEQ ID NO: 15 - protein, 9 aa, Homosapien
QRSGNNSVG

SEQ ID NO: 16 - protein, 27 aa, Homosapien
CAAAGATCAGGTAACAACTCCGTGGGT

SEQ ID NO: 17 - protein, 26 aa, Homosapien
TPTGTHPLAKKLPAQRTNRAVALSSS

SEQ ID NO: 18 - protein, 78 aa, Homosapien
ACACCCACGGGCACCCACCCCCTGGCAAAGAAGCTCCCTGCCCAGAGGACTAACAGGGCAGTGGCCTTGTCCAGCTCG

SEQ ID NO: 19 - protein, 18 aa, Homosapien
KENATTDLLTKLPAHTVG

SEQ ID NO: 20 - protein, 57 aa, Homosapien
TCCAAGGAGAACGCTACCACGGACCTCCTCACTAAGCTGCCTGCGCACACAGTGGGG

SEQ ID NO: 21 - protein, 24 aa, Homosapien
RGSEIVAGLEKMPARRASLSHPRD

SEQ ID NO: 22 - protein, 72 aa, Homosapien
CGAGGAAGCGAGATCGTGGCTGGACTGGAGAAGATGCCTGCCCGCCGGGCTTCCTTATCCCACCCCAGAGAC

SEQ ID NO: 23 - protein, 21 aa, Homosapien
KEVVSAQPATFLARSPHVGVK

SEQ ID NO: 24 - protein, 63 aa, Homosapien
AAGGAAGTGGTCTCTGCCCAGCCTGCCACCTTCCTGGCCCGTAGCCCTCACGTGGGTGTGAAG

(19) United States
(12) Patent Application Publication
(10) Pub. No.: US 2008/0199470 A1
(43) Pub. Date: Aug. 21, 2008

(54) The Use of Granulin-Epithelin Precursor (GEP) Anitbodies for Detection and Suppression of Hepatocellular Carcinoma (HCC)

(76) Inventors: Siu Tim Cheung, Hong Kong (HK); Chung Yee Jenny Ho, Hong Kong (HK); Sheung Tat Fan, Hong Kong (HK)

Correspondence Address: COOPER & DUNHAM, LLP, 1185 AVENUE OF THE AMERICAS, NEW YORK, NY, 10036, US

(21) Appl. No.: 11/946,260

(22) Filed: Nov. 28, 2007

Related U.S. Application Data
(63) Continuation-in-part of application No. 10/836,390, filed on Apr. 29, 2004.
(60) Provisional application No. 60/861,318, filed on Nov. 28, 2006.

(51) Int. Cl. A61K 39/395; G01N 33/53; G01N 33/566; A61P 1/00; G01N 33/574

(52) U.S. Cl. 424/138.1; 435/7.92; 435/7.23; 436/501

(57) ABSTRACT

This invention provides methods for detecting serum GEP level. This invention further provides methods for determining whether a subject is afflicted with Hepatocellular carcinoma (HCC) by measuring serum GEP level. In another embodiment, this invention provides methods for the suppression of HCC growth and progression both in vitro and in vivo by treating a patient with anti-GEP monoclonal antibody A23.

### CROSS-REFERENCE TO RELATED APPLICATIONS

This application is a continuation-in-part of U.S. patent application Ser. No. 10/836,390, filed Apr. 29, 2004. This application also claims priority of U.S. Provisional Patent Application No. 60/861,318, filed Nov. 28, 2006. The entire contents of each of the foregoing applications are incorporated herein by reference.

### FIELD OF THE INVENTION

This invention relates to Granulin-Epithelin Precursor (GEP) and methods which affect expression, translation, and biological activity of GEP in Hepatocellular Carcinoma (HCC). Another aspect of the invention relates to the detection methods of GEP, which are potential methods for diagnosis and treatment of HCC.

Several publications are referenced herein by Arabic numerals with parenthesis. Full citations for these references may be found at the end of the specification immediately preceeding the claims. The entire contents of these publications are incorporated by reference herein.

### BACKGROUND OF THE INVENTION

Liver cancer is the fifth most common cancer and the third leading cancer killer worldwide, and is responsible for about half million new cases and almost as many deaths per year (1,2). Hepatocellular carcinoma (HCC) is the major histological type of primary liver cancer. The major risk factor for developing HCC in Asia is hepatitis B virus (HBV) infection, whereas hepatitis C virus (HCV) infection is the major risk factor in Western countries and Japan. Prognosis for HCC patients afflicted by these cancers in general is worse with median survival duration less than a year, because the majority of these cancers are unresectable, not suitable for new treatment modalities, and have low chemotherapy response rates. Surgical resection, such as partial hepatectomy or liver transplantation, is the curative treatment for the disease (3-5). However, only 20% of patients are eligible for surgery because the majority of patients are diagnosed at an advanced stage with intra- and/or extra-hepatic metastases. After curative surgery, recurrence is common and the incidence is about 50% in the first year (6). Thus, early detection of HCC is essential to improve survival. The development of serological diagnostic tests for the detection of early-stage cancers in asymptomatic patients would be an important endeavor.

Currently, serum α-fetoprotein (AFP) has been widely used for HCC diagnosis (7). However, the serum AFP cutoff for detecting HCC in patients with coexisting liver diseases has not reached consensus with values ranging from 10 to 500 n/ml (8-10). The serum AFP test when used with the conventional higher cut-off point of 500 ng/ml revealed a sensitivity of about 50% and a specificity of more than 90% in detecting the presence of HCC in patients with coexisting liver disease (9). When used with lower cut-off values between 10 and 19 ng/ml, the sensitivity of the serum AFP test was 45% to 100% and with a specificity of 70% to 95% (10). Therefore, the identification of a novel biomarker with better sensitivity and specificity is urgently required for a better diagnosis of HCC.

Granulin-epithelin precursor (GEP) (SEQ ID No. 1 for nucleotide sequence and SEQ ID No. 2 for amino acid sequence) is an autocrine growth factor and belongs to a family of non-classical growth factors. Significant elevation of GEP mRNA level in HCC tissues was reported in our earlier cDNA microarray study (11). The inventors have further validated the observation in a separate patient cohort and confirmed that GEP protein is upregulated in HCC tissues but not in their adjacent non-tumor liver tissues (hepatitis and cirrhosis livers) and normal livers (12). Functional studies demonstrated that GEP controls HCC cell proliferation rate, invasion and metastasis in our earlier studies (12). As GEP is uniquely overexpressed and an important growth factor in HCC, the inventors hypothesized that the upregulation of GEP in HCC tumor tissues would also lead to an elevation of serum GEP protein level in HCC patients. Assay kit for detection of serum GEP is not available to the inventors knowledge, and therefore whether serum GEP levels have diagnostic significance have not yet been investigated.

The significant elevation of GEP in HOC and its function in enhancing cancer cell proliferation, makes GEP an attractive target for antibody therapy. In fact, targeted cancer therapy is promising to limit non-specific toxicity and to improve therapeutic efficiency, compared to chemotherapeutic agents with major drawback on lack of selectivity, severe side-effects, limited efficacy, and emergence/selection of drug-resistance (13). With the advance in hybridoma technology in the production of humanized and murine-human chimeric monoclonal antibody, targeted cancer therapy can be achieved by the use of the monoclonal antibody (14). Monoclonal antibody (mAb) therapy has proven efficacious in clinical cancer treatment, for example, anti-CD20 mAB (Rituximab) for B-cell lymphoma (15), anti-Her2 neu mAB (Herceptin) for metastatic breast cancer (16-17) and anti-EGFR and VEGF for metastatic colorectal cancer (18,19). However, development of targeted therapeutics, including antibody therapy, for HCC is limited, therefore, novel treatment target is urgently needed.

There is so far no report on the diagnostic significance of serum GEP in any human cancer. In this study, the inventors have determined the serum GEP levels in HCC patients, HBV chronic carriers and healthy individuals, to utilize GEP as a novel diagnostic marker for HCC. Moreover, the inventors have also examined the anti-tumor efficacy of their newly isolated anti-GEP mAb on human HCC of mouse xenograft model. It is demonstrated that anti-GEP mAbs are able to retard the growth of established tumor both in vitro and in vivo. These results indicate the potential application of anti-GEP mAbs in the treatment of HCC.

### SUMMARY OF INVENTION

The inventors have discovered that a protein, Granulin-Epithelin Precursor (GEP), is abundantly and uniquely expressed in heptocellular carcinoma (HCC), as compared to the surrounding normal liver tissue from HCC patients and normal liver tissue from healthy individuals.

It is an object of this invention to provide agents and methods for detecting GEP gene products in serum. It is also an object of this invention to provide agents and methods for sensitively detecting GEP gene products in serum of HCC patients for diagnostic purposes. Another object of this invention is to provide methods of producing GEP monoclonal and polyclonal antibodies with specific GEP peptide. Yet another object of this invention is to provide methods of producing anti-GEP monoclonal antibody (e.g. A23). A further object of this invention is to utilize anti-GEP monoclonal antibody (e.g. A23) for the suppression of HCC progression.

This invention further provides methods and strategies for determining GEP levels in HCC patients, hepatitis B carriers, and healthy objects.

To achieve the objects and in accordance with the purpose of the invention, as embodied and properly described herein the present invention provides agents, compositions and treatment of HCC in which exhibit altered expression of GEP or altered biological activity of GEP.

Use of the term “altered expression” herein means increased expression or overexpression of GEP or up-regulation of GEP protein as compared to corresponding normal cells or surrounding normal peripheral cells. The term “altered expression” also means expression which became unregulated or constitutive without being necessarily elevated. Use of the term “altered biological activity” herein means the change in activity of GEP that may or may not be dependence of GEP expression. The term “altered biological activity” also means a condition wherein change in any of the biological functions (e.g. proliferation, differentiation, metastasis) conferred by GEP results in the same or equivalent condition as altered expression of GEP.

Use of the term “GEP” herein means Granulin-Epithelin precursor in cellular extracts of HCC or cellular extracts of normal liver or extracellular fluids of HCC patients, or cellular extracts of liver or extracellular fluids of chronic hepatitis B carriers, cellular extracts of liver or extracellular fluids of healthy individuals.

Use of the term “neutralizing” herein means to counteract the activity or effect of GEP using the anti-GEP antibodies.

“Immunohistochemistry” described herein means the use of immuno-histochemistry method to detect the presence of GEP in the said HCC or normal liver or adjacent normal liver tissue samples. The term “immunohistochemistry” described herein also means a visualization method with the use of rabbit or mouse anti-human GEP polyclonal antibody and horseradish peroxidase (HRP)-conjugated goat-anti-rabbit or goat-anti-mouse secondary antibody and diaminobenzene (DAB) and hydrogen peroxide.

“Western Blot analysis” described herein means a method of separating extracted proteins from HOC samples by gel electrophoresis; transfer of separated protein samples onto a membrane; and detection of GEP with rabbit or mouse anti-human GEP antibody and horseradish peroxidase (HRP)-conjugated goat-anti-rabbit or goat-anti-mouse secondary antibody; and visualization of GEP with chemiluminescence techniques.

“Receiver operating characteristic (ROC) curve” described herein are for the examination of the performance characteristics of the GEP over their range. The area under the curve (AUC) is used as an index of global test performance, with a reference AUC of less than 0.5 indicating no discrimination ability.

All data described herein are analyzed by SPSS (version 11.0 for Windows, SPSS Inc., Chicago, Ill.). Categorical variables are compared using chi-square test or Fisher exact test where appropriate. Student's t-test is used for statistical comparison between two groups of continuous variables. Correlation is analyzed by Pearson correlation. Differences are considered significant when P<0.05.

Specific EXAMPLEs presented herein provide a description of preferred embodiment, particular the use of anti-GEP antibodies for detection and the use of neutralizing anti-GEP antibodies for inhibition of in vitro and in vivo GEP activities in HCC.

### BRIEF DESCRIPTION OF DRAWINGS AND FIGURES

FIG. 1 shows specificity of the GEP antibodies by Western blot analysis. (A) The monoclonal GEP antibody A23 specifically recognized the GEP-glycosylated form ˜88 kDa from the cell lysate of HepG2 (G2) and Hep3B (3B), and recombinant GEP-full length (FL). GEP was significantly upregulated in the tumor (T) compared to its adjacent non-tumor liver tissue (N) (patients 289 & 291). (B) Immunoprecipitation from hepatoma cell lysate Hep3B (3B), HepG2 (G2) and Huh7 (H7). Lanes 1, 3 and 5 were immunoprecipitation using monoclonal GEP antibody A23. Lanes 2, 4 and 6 were mock immunoprecipitation using mouse IgG. The rabbit polyclonal GEP antibody was used for detection. Lanes 7, 8 and 9 were cell lysate from the same hepatoma cell lines. The GEP at ˜88 kDa from the A23 immunoprecipitated complex confirmed the specificity of the monoclonal and polyclonal antibodies. (C) Detection of secretory GEP in the supernatants of cultured hepatoma cells Hep3B (3B), HepG2 (G2) and Huh7 (H7) in lanes 1, 2 and 3, respectively. Lanes 4, 5 and 6 were cell lysate from the same hepatoma cells.

FIG. 2 shows localization of GEP in human liver tissues. (A) Expression of GEP (visualized as brown stain) was detected in the neoplastic hepatocytes but not in other cell types of the tumor components (400× magnification). (B) Tumor adjacent non-tumor liver tissues (400× magnification) revealed no GEP signal in non-neoplastic hepatocytes.

FIG. 3 shows concentration of serum GEP in 72 healthy donors, 38 patients with chronic hepatitis B and 107 HCC patients.

FIG. 4 shows receiver-operating characteristic curve analysis on serum GEP performance (bold solid line). “Sensitivity” (true positive fraction) was plotted against “1-Specificity” (false positive fraction).

FIG. 5 shows in vitro treatment with A23 led to cell growth inhibition in a dose-dependent manner. Cell proliferation was measured via MTT assay. A) HepG2 cells and B) Hep3B cells were incubated with PBS (control) (▪), A23-50 μg/ml [symbol] or A23-100 μg/ml () in presence of 1% FBS for 5 days. Compare with the PBS control, differences were significant at *P<0.05 level. C) GEP concentration in HepG2 and Hep3B culture supernatant after A23 treatment (+) or PBS Control (−) was measured by direct ELISA. D) A23 treatment of Hep3B and HepG2 led to a decrease in MAPK phosphorylation. HCC cell lines were serum-starved for 24 hours and then treated with A23-100 μg/ml (lane 1-HepG2 and lane 3-Hep3B) or PBS (control) (lane 2-HepG2 and lane 4-Hep3B) for 72 hours. Cell lysates (10 μg) were immunoblotted with rabbit polyclonal GEP, anti-phospho-MAPK and anti-MAPK antibodies, anti-β-actin was used as a control for protein loading and transfer.

FIG. 6 shows growth inhibition of Hep3B tumor xenografts in nude mice. Dose-dependent effects for treatment of established Hep3B tumor treated with A23 on a twice weekly schedule. A23 antibody was injected intraperitoneally at A23-50 μg [symbol] or A23-100 μg () and PBS were used as control (▪). Compare with the PBS control, differences were significant at *P<0.05 and **P<0.005 level.

FIG. 7 shows serum profile of mice at day 31 after A23 treatment. A) A23 concentration. B) GEP concentration.

FIG. 8 shows A) Histologic examination of Hep3B xenografts at day 31 after A23 treatment at 200× magnification. B) Histologic examination of non-tumor liver at day 31 after A23 treatment at 200× magnification.

FIG. 9 is an analysis of proliferation and apoptosis effect of A23 in Hep3B tumors. A) Proliferation of tumor cells in xenografts was evaluated by Ki-67 staining. B) Apoptosis of tumour cells was evaluated by TUNEL assay.

FIG. 10 shows the effect of A23 on Hep3B xenografts. Total xenograft lysate (20 μg) was immunoblotted with the indicated phosphospecific antibodies to phospho-p44/42 MAPK (Thr202/Tyr04) and phospho-AKT (Ser473) antibody. Total MAPK and AKT were used as loading control. Anti-GEP blot was also shown and a representative β-actin reprobed blot is shown as loading control. Xenografts from PBS control treatment mice (Lane 1), 50 μg A23 treated (Lane 2) and 100 μg A23 treated (Lane 3).

### DETAILED DESCRIPTION OF THE PREFERRED EMBODIMENTS

Reference will now be made in detail to the presently preferred embodiments of the invention, which, together with the examples and figures following the detailed description, serve to explain the principles of the invention.

From earlier cDNA microarray analysis (11), the inventors identified GEP as a potential tumor marker of HCC. The inventors have further validated the observation in a separate set of patient samples and confirmed that GEP protein is upregulated in HCC tissue (12). In addition, the inventors have also shown that GEP level positively regulates cancer cell proliferation and tumor invasiveness (12). As GEP is a secretory autocrine growth factor, the inventors hypothesized that the upregulation of GEP in HCC tumor tissues would also lead to an elevation of serum GEP protein level in patients and hence act as a useful diagnostic marker for the disease.

In the present study, the inventors report the generation of GEP specific monoclonal and polyclonal antibodies. Using the newly isolated monoclonal antibody, GEP protein level was shown to be upregulated in HCC tumor tissues, which is in agreement with previous observation (11,12). From immunohistochemical study, GEP protein was expressed in the neoplastic hepatocytes but not the other tumor components. The inventors then evaluated if GEP protein would be secreted from HCC cells, by performing immunoblotting from HCC cell line conditioned medium. The inventors have shown that GEP was detectable from the culture supernatant, suggesting that GEP could be a secretory protein detectable in HCC patient sera.

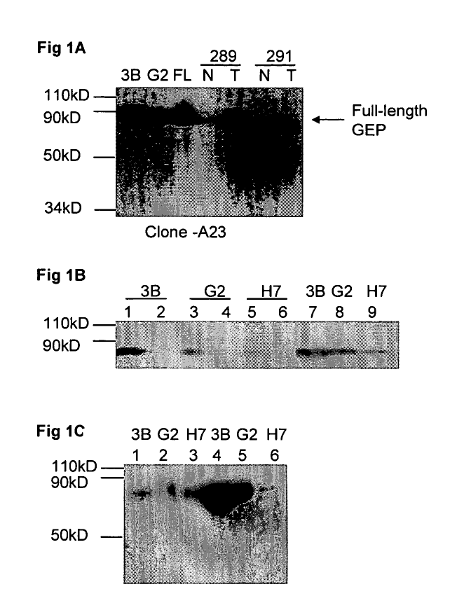

To detect the GEP serum protein, a specific GEP ELISA has been established using the newly isolated antibodies. Monoclonal antibody targeting the C-terminus of GEP was used as the capture antibody and a polyclonal antibody targeting the center part of GEP as the detection antibody. The use of these antibodies combination which target two different epitopes of the GEP full-length protein enhanced the specificity of the assay as confirmed by the immunoprecipitation experiment (FIG. 1B).

Nonetheless, due to the heterogeneity of HCC (20), it is questionable if there would be a tumor marker that expressed in all HCC tissues. However, the combination use of two to three markers will enhance the sensitivity of detection. In the current study, the inventors demonstrated that serum GEP level has no correlation with serum AFP level in HCC patients. Sensitivity of HCC diagnosis by either one marker was only 58.0% (AFP alone) to 60.7% (GEP alone), but the sensitivity increased to 87.9% by combination use of these two markers.

The high fatality-to-case ratio associated with HCC is partially caused by the lack of symptoms in its early stages. Curative resection can only be the treatment of choice for 20% of HCC patients. Early detection of HCC is therefore essential to improve survival. In the current study, serum GEP was also detectable in early-stage HCC patients (56.6%), suggesting this maker would be useful for early diagnosis which is important to improve patient survival. Thus, serum GEP determination would enhance early detection of HCC, allowing for better treatment option and survival outcome.

The inventors previously have shown that the down-regulation of GEP using the antisense approach can significantly reduce the tumorigenicity of HCC in athymic nude mice model (12). This observation suggested that GEP is an attractive target for cancer therapy. However, the mode of gene delivery and infection/transfection efficiency remains as the main obstacle in successful cancer gene therapy. The use of GEP antibody compare to gene therapy is a more practical and feasible approach for targeted cancer therapy. As GEP is a secretory autocrine growth factor, therefore the inventors hypothesized that neutralizing the extracellular GEP by GEP-specific antibody A23 may hinder the proliferation function of GEP. Unlike targeting by antisense approach, antibody targeted therapy, like Herceptin and anti-VEGF, has higher efficacy and lower toxicity and make targeted therapy feasible in cancer patients.

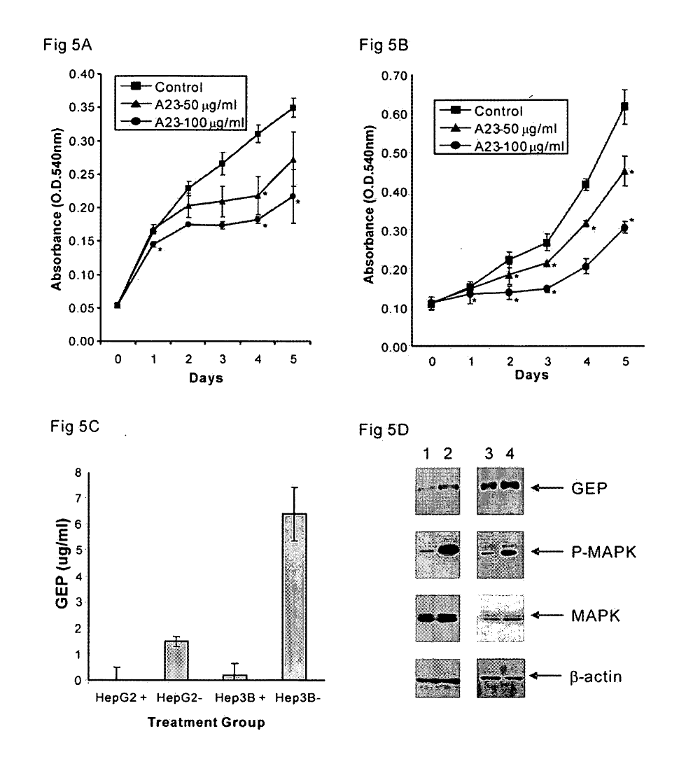

In order to investigate the inhibitory effect of anti-GEP antibodies, e.g. A23, they were added to culture supernatant of HepG2 and Hep3B cells in the presence of 1% FBS. Proliferation of the cancer cells were significantly inhibited by the mAbs A23 when compare to no treatment control in a dose dependent manner (FIGS. 5A and 5B). Concentration of GEP in the culture supernatant was measured by sandwiched ELISA. Hep3B has a higher concentration of GEP in the culture supernatant than HepG2 (FIG. 5C). After 72 hours of A23 treatment, the concentrations of GEP in the culture supernatant were reduced in both cell lines (FIG. 5C). This result indicated that addition of A23 could effectively neutralize the GEP secreted into the culture supernatant. GEP has been shown to stimulate the phosphorylation of p44/42 mitogen activate protein kinase (MAPK) in the extracellular regulated kinase signaling pathway. To investigate whether the inhibition of proliferation by anti-GEP treatment is related to the phosphorylation of p44/42 MAPK, Western blot analysis was performed on cultured cell lysate after treatment with A23. As shown in FIG. 5D, the addition of anti-GEP A23 in the culture supernatant for 72 hours significantly reduced the phosphorylation of MAPK in both HepG2 and Hep3B cell lines suggesting that the reduction of cell proliferation is dependent on the reduced phosphorylation of p44/42 MAPK.

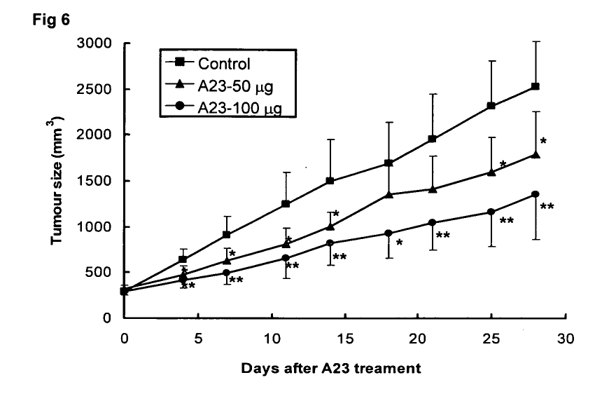

In animal study, the antitumor effect of anti-GEP mAbs A23 was confirmed with Hep3B tumor implanted on nude mice. Antibody treatment of 50 and 100 μg/injection was started once the tumor size reached ˜0.3 cm³. Nine doses of treatments were given twice a week and the tumor sizes were monitored. After 5 weeks of treatment, the median tumor volume of mice treated with anti-GEP A23 were 1.57 cm³ (range 1.44-2.53 cm³) and 1.21 cm³ (range 0.79-1.97 cm³) for 50 μg and 100 μg treatments, respectively, whereas that of the median tumor volume of the control mice was 2.20 cm³ (range 1.65-3.04 cm³). Analysis of variance by t-test demonstrated that difference between treated and untreated animal were statistically significant (P<0.05) (FIG. 6). This experiment indicated that in objects treated with A23 resulted in a dose-dependent suppression of Hep3B tumor growth. Moreover, this model mimics the situation in the clinic when most HCC patients were diagnosed at late stage and become in-operable. The marked decrease in tumor volume from the antibody treatment, suggested that neutralizing GEP using anti-GEP antibody can significantly delay tumor proliferation even in an established tumor. The current study demonstrated that anti-GEP therapy is feasible for stabilizing the disease and/or delay tumor progression.

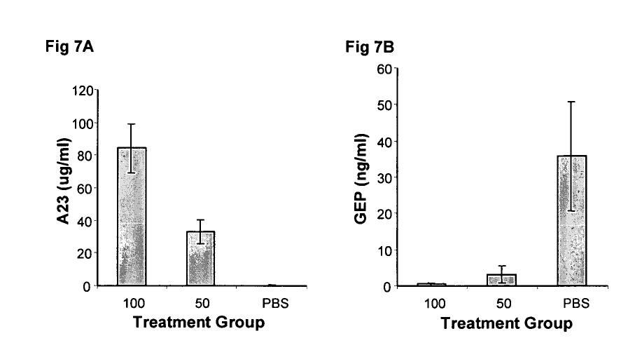

As the anti-GEP mAbs A23 was injected intraperitoneally, the antibody titer was measured in order to evaluate the actual amount of antibody found in the mice blood circulation. The antibody titer of anti-GEP mAbs A23 in the mice serum were measured by direct ELISA. As expected, the level of A23 in the control group was undetectable, but remained high in treatment group. For the 100 μg treatment group, the median level of A23 was 74.61 μg/ml (range from 4.50 μg/ml to 145.48 μg/ml). For the 50 μg treatment group, the median level of A23 was 8.87 μg/ml (range from 1.35 to 16.24 μg/ml) (FIG. 7A). In order to examine the effectiveness of A23 in the clearance of serum GEP, the concentration of GEP in mice serum was measured by sandwiched ELISA. For the PBS control group, the serum GEP level was highest with the median level of GEP of 21.46 ng/ml (ranged from 8.33 to 137.50 ng/ml). However, after A23 treatment, the serum GEP level was significantly lowered (P<0.05). After 100 μg treatment, the serum GEP level was barely detectable (median=0 ng/ml, range from 0 to 2.5 ng/ml). After 50 μg treatment, the median level of GEP was reduced to 7.08 ng/ml (range from 0 to 10.83 ng/ml) (FIG. 7B).

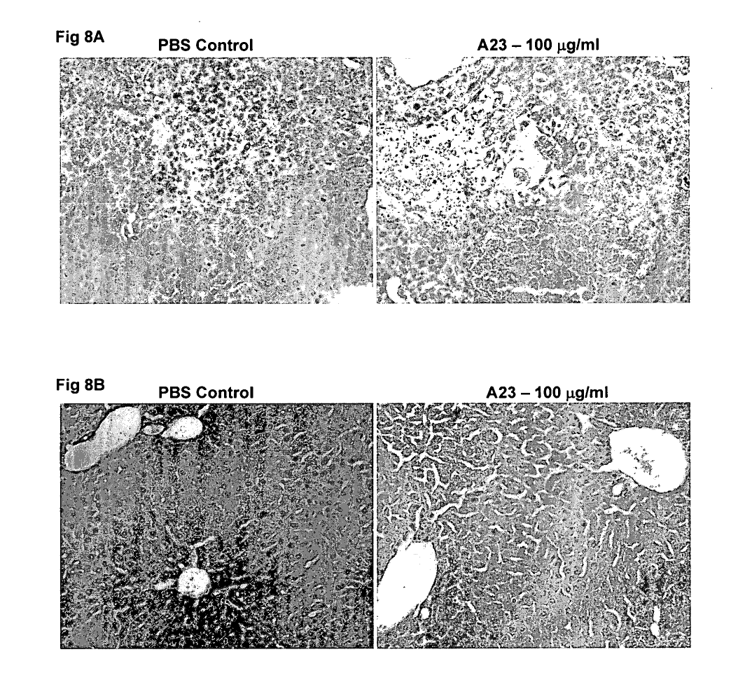

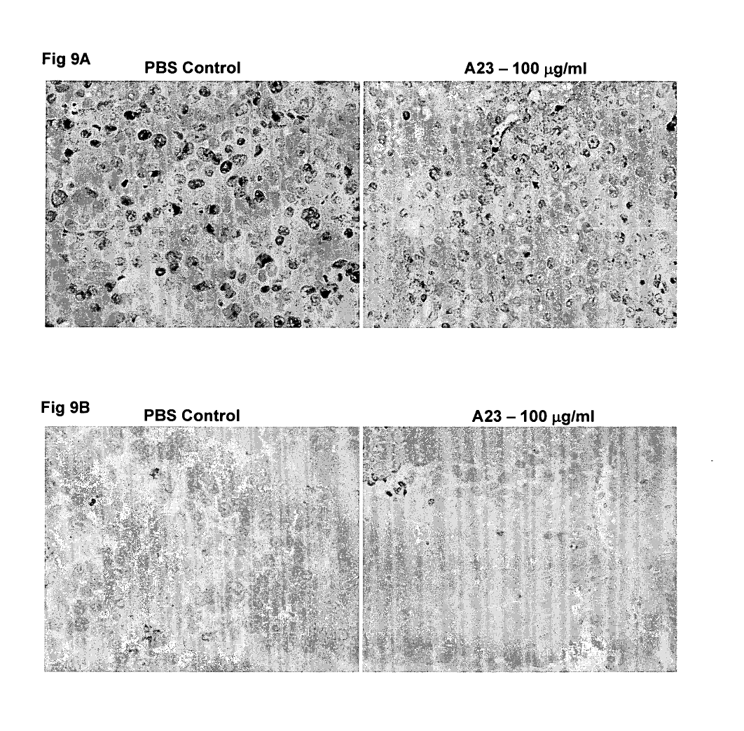

Histologic examination of xenografts at the end of the treatment showed marked difference in the tumor from animals receiving A23 compared with tumor from animals receiving control therapy. In the 100 μg A23-treated group, massive necrotic areas were found and there were substantially more cell-sparse regions compared with the control group (FIG. 8A). There was no gross histological difference in the non-tumor liver from the treatment and control group (FIG. 8B).

Immunohistological examination of xenografts was performed using Ki-67 antibody, there was a marked decrease in Ki-67 positive cells in 100 μg A23-treated mice compared to the control group (FIG. 9A). However, there was no difference in the number of positive cell from the TUNEL assay in the treatment and control group (FIG. 9B). These results indicated that the decrease in tumor volume by the A23 treatment was caused mainly by a decrease in proliferation but not an increase in apoptosis.

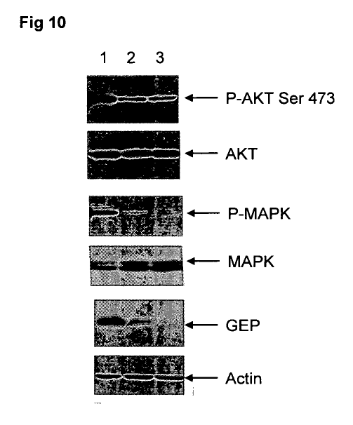

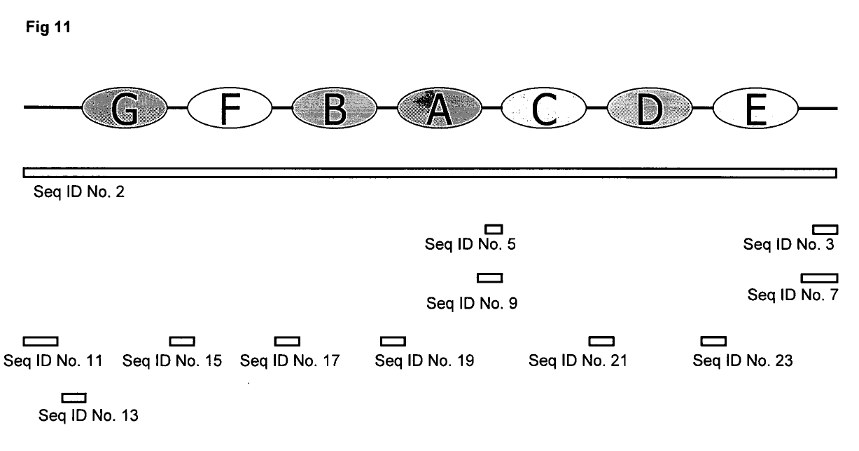

To investigate the mechanism of anti-GEP antibody actions on tumor cell proliferation in mouse xenograft, the phosphorylation level of the key proliferative gene, MARK and AKT were examined. The phosphorylation of both MAPK and AKT at Ser473 were significantly reduced upon anti-GEP treatment suggesting that anti-GEP antibody treatment delay tumor cell proliferation via the MAPK and AKT pathway (FIG. 10). These observations showed that anti-GEP delay tumor cell proliferation both in vitro and in vivo. It inhibited p44/42 MAPK phosphorylation and AKT phosphorylation in a dose dependent manner.

In summary, the inventors have shown that GEP is a novel serum marker of HBV-related HCC. The combination of AFP and GEP improves the diagnostic sensitivity of HCC in both early-stage and late-stage tumors. The availability of this simple and reliable immunoassay for measuring serum GEP concentration may provide a valuable tool to further evaluate the clinical usefulness of serum GEP for the management of HCC. Furthermore, the inventors have shown that anti-GEP antibodies are able to inhibit the growth of established HCC tumors. These results indicated that GEP is a target for HCC therapy and the potential application of anti-GEP antibodies for treatment of HCC.

### Example 1

### Patient Specimens

The study protocol was approved by the Institutional Review Board of The University of Hong Kong and signed consents were obtained from the patients and controls. Between March 1999 and October 2004, blood samples were obtained from 107 patients diagnosed with primary HCC, 38 chronic hepatitis B patients (only those with no indication of malignancy for more than 2 years of follow-up were included in the current study) and 72 healthy donors who were hepatitis B surface antigen (HBsAg) negative. Serum HBsAg was positive in 96 (89.7%) HCC patients, and therefore control groups included chronic hepatitis B patients and healthy volunteers. Serum samples were frozen at −70° C. until use. Tumor and adjacent non-tumor liver tissues from HCC patients were collected and snap frozen in liquid nitrogen and stored at −70° C. until use. Parallel sections were formalin-fixed and paraffin embedded for histological examination and immunohistochemical study. Clinical and pathological data including the serum AFP level of all patients and control subjects were prospectively collected.

### Example 2

### Cell Lines

The human HCC cell lines Hep3B, HepG2 and Huh7 (American Tissue Culture Collection, Manassas, Va.) and Japan Health Science Research Resources Bank, Osaka, Japan) were maintained in Dulbecco's modified Eagle medium (DMEM) supplemented with 10% fetal bovine serum (Gibco BRL, Carlsbad, Calif.).

### Example 3

### Establishment of Antibodies

GEP-specific antibody was generated by immunizing BALB/c mice with 33 μg of Keyhole Limpet Hemocyanin (KLH)-conjugated custom-made GEP specific peptide SEQ ID No:3 subcutaneously with complete Freund's adjuvant (Sigma-Aldrich, Dorset, UK). For subsequent booster, the same amount of antigen was injected intraperitoneally in incomplete Freund's adjuvant biweekly. Serum antibody activity to the immunizing antigen was monitored after each boost using ELISA against peptide antigen. Mice showing high serum antibody titer to the antigen were given a final boost of intravenously injected antigen 3 days prior to harvesting the spleens.

### Generation of Anti-GEP Monoclonal Antibody A23

Spleen was harvested from mice shown high titre of antibody in their serum. Fusion of the spleen cells with a nonproducer myeloma line, NS0, was carried out according to the standard protocols originally derived from Kohler and Milstein (21). NS0 was maintained in DMEM supplemented with 10% fetal bovine serum (Gibco BRL, Carlsbad, Calif.). Briefly, lymphocytes were harvested from the mouse spleen and fused with NS0 using Polyethylene Glycol 1500 (Roche Diagnostics GmbH, Mannheim, Germany). Hybridoma was selected by plating into DMEM medium contained HAT and 20% FBS. Antibody secreting hybridoma were selected by ELISA and subsequently subcloned by limited dilution. Isotypes of the monoclonal antibody were determined using the Mouse MonoAB ID Kit (HRP) (Zymed Laboratories, Inc., San Francisco, Calif.).

### Development of Polyclonal Antibodies Against GEP

New Zealand white rabbits were immunized with 100 μg of Keyhole Limpet Hemocyanin (KLH)-conjugated GEP specific peptide SEQ ID No:4 (Zymed Laboratories, Inc., San Francisco, Calif.) using standard procedures (22) The rabbit antisera were affinity purified using the immobilized antigen column, dialysed against 1×PBS and concentrated to 1 mg/ml.

### Generation and Verification of Monoclonal Antibodies

To generate the GEP monoclonal antibodies, a synthetic peptide of 16-amino acid, SEQ ID NO:3, designated at the GEP carboxyl-terminal was used as an immunogen to generate the antibodies. The clones were then subjected to another round of ELISA screening against full-length recombinant GEP and Hep3B cell lysate. The supernatants of these clones were then subjected to Western blot analysis and subcloned by limited dilution. Clone A23 was identified, as the only antibody that recognized the GEP glycosylated form at 88-Kda from the GEP recombinant protein (FL), HCC cultured cell lysate (Hep3B and HepG2) and patients' tissue lysate (FIG. 1A). To increase the specificity of the sandwiched ELISA against full-length GEP, the inventors custom-made another GEP specific polyclonal antibody specifically recognizing the center parts of GEP, SEQ ID NO:4.

To determine the specificity of the polyclonal and monoclonal GEP antibodies, immunoprecipitation was performed. The monoclonal and polyclonal GEP antibodies recognized the 88-kDa glycosylated GEP from the culture lysate (FIG. 1B).

To determine whether GEP was a secretory protein, GEP was examined in the conditioned medium from the HCC cell lines using the GEP monoclonal antibody. As shown in FIG. 1C, the 88-kDa glycosylated GEP was detectable in the supernatant of HCC cells.

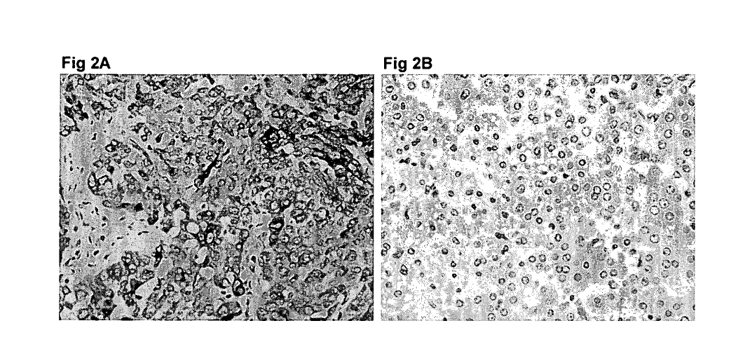

GEP localization was revealed by immunohistochemistry on tumor tissue paraffin sections. The protein signals were found to be uniformly associated with neoplastic hepatocytes but not in the endothelial cells or fibroblasts in the tumor tissues, while hepatocytes in the non-tumor tissues revealed no signals (FIG. 2).

### Example 4

### Protein Extraction, Western Blotting and Immunoprecipitation

HCC cell lines, HCC and adjacent non-tumor liver tissues were subjected to Western blot analysis. Total proteins were extracted by homogenizing snap frozen patients' samples in Buffer A (8 M Urea, 50 mM Tris-HCl pH 8.0) containing 1 mM PMSF. Protein extracts, totally 10 μg, were separated by 10% SDS-PAGE gel followed by Western blotting. The blot was blocked with 5% skim milk in PBS/0.1% Tween 20 and probed with the appropriate monoclonal antibodies. Polyclonal goat anti-β-actin antibody was used as 1:1000 dilution (DAKO, Glostrup, Denmark). Secondary anti-mouse and anti-goat horseradish peroxidase (HRP) conjugated antibodies respectively were used in 1:3000 dilution (AP biotech, Chalfont St. Giles, UK). ECL was performed according to the manufacturer's instructions (AP biotech, Chalfont St. Giles, UK). Immunoprecipitation was performed with 500 μg of cell lysate and incubated with 1 μg of the purified monoclonal antibodies. The immunocomplexes were separated on an SDS-PAGE and immunoblotted with the polyclonal anti-GEP antibody.

### Example 5

### Immunohistochemistry

Immunohistochemistry study was performed on paraffin-embedded HCC and adjacent non-tumor liver tissues. Protocol was described previously with modification (12). Antigen retrieval was performed by microwave with sections immersed in citrate buffer, followed by endogenous peroxidase blocking and biotin blocking reagents (DAKO, Glostrup, Denmark). Appropriate monoclonal antibodies were used as 2 μg/ml. Signal was detected by anti-mouse HRP conjugated secondary antibody and color development with diaminobenzidine (DAB) as the chromogen. Tissue sections were counterstained with hematoxylin.

### Example 6

### Determination of GEP Levels in Subject Serum

Ninety-six-well ELISA plates (Nalge Nunc International, Rochester, N.Y.) were coated with 0.5 μg of anti-GEP mAb A23 in 50 μl of PBS per well. The plates were blocked for 1 hour with 300 μl of blocking buffer (1×PBS, 1% BSA, 5% Surcose, 0.05% NaN₃), then 50 μl of 1:5 diluted serum samples was added and incubated at room temperature for 2 hours. After washing the unbound material with 0.05% Tween 20 in 1×PBS, bound GEP was detected using an affinity purified anti-GEP rabbit polyclonal antibody (1:2000, 1 mg/ml) followed by incubation with horseradish peroxidase-conjugated goat anti-rabbit IgG (Zymed Laboratories, Inc, San Francisco, Calif.) using TMB (Pierce Biotechnology Inc., Rockford, Ill.) as substrates. To quantify the GEP present in the serum, a calibration curve of purified GEP diluted in PBS with 10% Fetal Bovine Serum was performed in parallel. Each sample was measured 3 times by quadruplicates. The dynamic range of the GEP sandwich ELISA was 469 pg/ml to 30 ng/ml. A pooled serum sample of patients was included in each assay and used for adjustment of plate-to-plate variation. The variations within and between assays were 2.9% (range 1.1-5.5%) and 5.0% (range 1.3-10.8%), respectively.

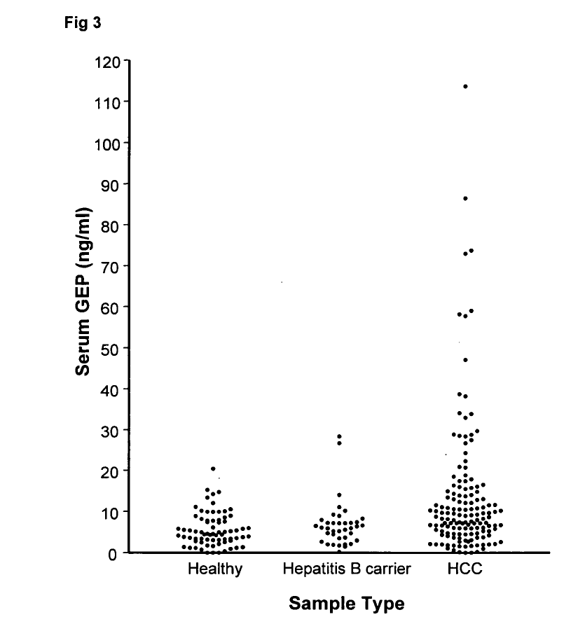

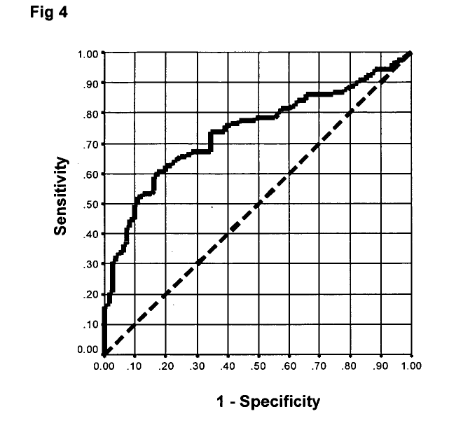

The serum GEP protein levels were measured by a specific ELISA in 107 HCC patients, 72 healthy individuals and 38 patients with chronic hepatitis B (FIG. 3). The median and mean levels of serum GEP in healthy subjects were 4.59 ng/ml and 5.63 ng/ml, respectively (range, 0 to 20.46 ng/ml). The median and mean concentrations of serum GEP in patients with chronic hepatitis B were 6.03 ng/ml and 6.85 ng/ml, respectively (range, 0.17 to 28.36 ng/ml). The median and mean serum GEP levels in HCC patients were 10.53 ng/ml and 16.09 ng/ml, respectively (range, 0 to 113.59 ng/ml). The serum GEP levels measured in HCC patients were significantly higher than those in healthy controls (P<0.001) and patients with chronic hepatitis B (P<0.001). An ROC curve for GEP was also constructed (FIG. 4), showing an AUC of 0.74 (95% Cl 0.67-0.81, P<0.001). To discriminate HCC from controls including chronic hepatitis B carriers and healthy individuals, the Youden index was employed to determine the optimal cutoff for class prediction. The optimal cutoff value was 9.07 ng/ml, which achieved a sensitivity and specificity of 60.7% and 82.5%, respectively.

### Example 7

### Diagnosis of HCC with Combined Screenings of Serum AFP and GEP

Serum AFP levels were also measured in the same set of samples and compared with the serum GEP data. When using serum AFP levels for HCC diagnosis, the cutoff value of 100 ng/ml was used which was considered as relatively high and specific (Tables 1 and 2). A lower cutoff value of serum AFP at 20 ng/ml was also examined and data in comparison with serum GEP was presented in the Supplementary Tables 1 and 2. The sensitivity of HCC diagnosis by serum AFP (58.0%, 62/107, cutoff at 100 ng/ml) and serum GEP (60.7%, 65/107, cutoff at 9.07 ng/ml) was comparable (Table 1). There was no correlation between GEP and AFP serum levels (r=−0.113; P=0.243) in HCC patients. The majority of HCC patients (87.9%, 94/107) demonstrated elevation of either serum GEP (>9.07 ng/ml) or AFP (>100 ng/ml). Importantly, the simultaneous use of these two markers increased the sensitivity of HCC diagnosis from 58.0% (elevation of AFP alone) to 87.9% (elevation of either AFP or GEP, or both).

### Example 8

### Early Diagnosis of HCC with Combined Screenings of Serum AFP and GEP

Early diagnosis is the key to enable HCC patients to receive curative treatment and to improve survival. The performance of the serum markers were examined according to tumor stages. In early-stage HCC patients, the sensitivity of detection by serum GEP (56.6%, 43/76) and serum AFP (55.3%, 42/76) was similar (Table 2). In late-stage patients, the sensitivity of HCC detection by serum GEP (71.0%, 22/31) was slightly better than serum AFP (64.5%, 20/31). Elevation of either serum GEP or AFP was observed in 84.2% (64/76) of early-stage patients and 96.8% (30/31) of late-stage HCC patients. Thus, the use of two markers would increase the sensitivity of diagnosis in both the early-stage and late-stage HCC patients.

### Example 9

### Cell Proliferation Assay

Cellular proliferation was measured via 3-(4,5-dimethylthiazol-2.yl)-2,5-diphenylthtrazolium bromide (MTT) assay. Briefly, 5×10³ cells were seeded to a 96-well plate in 100 μl DMEM medium containing 1% FBS either with or without mABs A23 as indicated. For every 24 hours, the medium was replaced with 100 μl DMEM containing 0.5 mg/ml MTT and incubated for 3 hours at 37° C. Crystal was dissolved by 100 μl MTT solvent (0.1N HCl in isopropanol) and absorbance was plot as the measurement at 540 nm subtracted the background absorbance at 650 nm. Each data point represented results from 3 independent experiments, each performed in triplicates.

Anti-GEP mAbs A23 was added to culture supernatant of HepG2 and Hep3B cells in the presence of 1% FBS, proliferation of the cancer cells were significantly inhibited by the mAbs when compare to no treatment control (FIGS. 5A and B). This inhibition is in a dose dependent manner (FIG. 5B). Concentration of GEP in the culture supernatant was measured by sandwiched ELISA. Hep3B has a higher concentration of GEP in the culture supernatant than HepG2 (FIG. 5C). After 72 hours of A23 treatment, the concentrations of GEP in the culture supernatant were reduced in both cell lines (FIG. 5C). This result indicated that addition of A23 could effectively neutralize the GEP secreted into the culture supernatant.

### Example 10

### Effect of Anti-GEP Antibody Treatment on the Phosphorylation of MAPK

Total proteins were extracted by homogenizing mouse xenografts and Hep3B cells in cell lysis buffer (Cell Signaling Technology Inc., Beverly, Mass.) containing 1 mM PMSF. Protein extracts, totally 10 g, were separated by 10% SDS-PAGE gel followed by Western blotting. The blot was blocked with 5% skim milk in PBS/0.1% Tween 20 and probed with the appropriate antibodies. Polyclonal goat anti-β-actin antibody was used as 1:1000 dilution (DAKO, Glostrup, Denmark). Polyclonal rabbit anti-GEP antibody was used as 1:500 dilution (12). Antibody against p44/p42 MAPK and phospho-p44/42 MAPK (Thr202/Tyr204) were used according to manufacturers' instruction (Cell Signaling Technology, Inc., Beverly, Mass.). Secondary anti-mouse, anti-rabbit and anti-goat HRP conjugated antibodies were used in 1:3000 dilution respectively (AP biotech, Chalfont St, Giles, UK). ECL was preformed according to manufacturer's instructions (AP biotech, Chalfont St. Giles, UK).

GEP has been shown to stimulate the phosphorylation of p44/42 mitogen activate protein kinase (MAPK) in the extracellular regulated kinase signaling pathway (23). To investigate whether the inhibition of proliferation by anti-GEP treatment is related to the phosphorylation of p44/42 MAPK, Western blot analysis was performed on cultured cell lysate after treatment with A23. As shown in FIG. 5D, the addition of anti-GEP A23 in the culture supernatant for 72 h ours significantly reduced the phosphorylation of MAPK in both HepG2 and Hep3B cell lines suggesting that the reduction of cell proliferation is dependent on the reduced phosphorylation of p44/42 MAPK.

### Example 11

### HCC Xenografts and Treatment of Subcutaneous Xenografts in Nude Mice

This study protocol was approved by the Committee on the Use of Live Animals in Teaching and Research of the University of Hong Kong. Mice (n=15) were housed in barrier facilities that provided 12-hour light-dark cycles and received food and water. All manipulations were performed while mice were under isoflurance gas anesthesia. No mouse showed signs of wasting or other signs of toxicity. Hep3B cells (2×10⁶ cells/mouse) were injected subcutaneous to 5- to 6-week-old male athymic nude mice. Tumor sizes were determined by Vernier caliper measurements and the tumor volume was calculated according to the formula (a×b²)/2, where a and b are the largest and smallest diameters respectively (24). Treatments were started as the tumor size reaches a mean tumor volume of ˜0.3 cm³ and mice were randomized into 3 groups (n=5). Antibodies were injected intraperitoneally twice weekly for the duration of the study. From our preliminary study, the half-life time (T_{1/2}) of serum A23 antibody in the mice was longer than 72 hours after intraperitoneal injection (data not shown), therefore a treatment regime of 100 μg and 50 μg intraperitoneally twice weekly was chosen. Group 1 mice were treated with either 100 μg purified mouse IgG (Zigma-Aldrich, Saint Louis, Mo.) or PBS. In preliminary studies, the inventors found no difference between mouse IgG or PBS on tumor growth. Group 2 and 3 mice were treated with 50 μg and 100 μg A23 mAbs, respectively.

The antitumor effect of anti-GEP mAbs A23 was examined on Hep3B tumor implanted on nude mice. Antibody treatment of 50 and 100 μg injection was started once the tumor size reached ˜300 mm³. Nine doses of treatments were given twice a week and the tumor sizes were monitored. After 5 weeks of treatment, the median tumor volume of mice treated with anti-GEP A23 was 1.57 cm³ (range 1.44-2.53 cm³) and 1.21 cm³ (range 0.79-1.97 cm³) for 50 μg and 100 μg treatments, respectively, whereas the median tumor volume of the control mice was 2.20 cm³ (range 1.65-3.04 cm³). Analysis of variance by t-test demonstrated that difference between treated and untreated animal were statistically significant (P<0.05) (FIG. 6). Treatment with A23 resulted in a dose-dependent suppression of Hep3B tumor growth.

### Example 12

### Quantification of GEP in Mice Serum after A23 Treatment

Mice serum was collected for measurement of antibody concentration and serum GEP concentration using ELISA.

Since the anti-GEP mAbs A23 was injected intraperitoneally, the antibody titer was measured in order to evaluate the actual amount of antibody found in the mice blood circulation. The antibody titer of anti-GEP mAbs A23 in the mice serum were measured by direct ELISA. As expected, the level of A23 in the control group was undetectable, but remained high in treatment group. For the 100 μg treatment group, the median level of A23 was 74.61 μg/ml (range from 4.50 μg/ml to 145.48 μg/ml). For the 50 μg treatment group, the median level of A23 was 8.87 μg/ml (range from 1.35 to 16.24 μg/ml) (FIG. 7A).

In order to examine the effectiveness of A23 in the clearance of serum GEP, the concentration of GEP in mice serum was measured by sandwiched ELISA. For the PBS control group, the serum GEP level was highest with the median level of GEP of 21.46 ng/ml (ranged from 8.33 to 137.50 ng/ml). However, after A23 treatment, the serum GEP level was significantly lowered (P<0.05). After 100 mg treatment, the serum GEP level was barely detectable (median=0 ng/ml, (range from 0 to 2.5 ng/ml). After 50 mg treatment, the median level of GEP was reduced to 7.08 ng/ml (range from 0 to 10.83 ng/ml) (FIG. 7B).

### Example 13

### Euthanasia and Processing of Tissue

Mice were euthanized by the end of 5 weeks. Xenografts and liver tissues were collected and snap frozen in liquid nitrogen and stored at −70° C. until use. Parallel sections were formalin-fixed and paraffin embedded for histological examination and immunohistochemical study.

Histologic Examination of Xenografts after A23 Treatment

Histologic examination of xenografts at the end of the treatment showed marked difference in the tumor from animals given A23 compared with tumor from animals receiving control therapy. In the 100 mg A23-treated group, massive necrotic areas were found and there were substantially more cell-sparse regions compared with the control group (FIG. 8A). There was no gross histological difference in the non-tumor liver from the treatment and control group (FIG. 8B).

Immunohistological examination of xenografts was performed using Ki-67 antibody, there was a marked decrease in Ki-67 positive cells in 100 mg A23-treated mice compared to the control group (FIG. 9A). However, there was no difference in the number of positive cell from the TUNEL assay in the treatment and control group (FIG. 98). These results indicated that the decrease in tumor volume by the A23 treatment was caused mainly by a decrease in proliferation but not an increase in apoptosis.

### Example 14

### Effect of Anti-GEP Antibody Treatment In Vivo

To investigate into the mechanism of A23 on cell proliferation, the phosphorylation level of the key proliferative protein, MAPK and AKT were examined using the total protein lysate from mouse tumor xenograft after treatment. Antibody against p44/p42 MAPK, phospho-p44/42 MAPK (Thr202/Tyr204), AKT and phospho-AKT(ser473) were used according to manufacturers' instruction (Cell Signaling Technology, Inc., Beverly, Mass.). The phosphorylation of both MAPK and AKT at Ser473 were reduced upon anti-GEP treatment (FIG. 10), suggesting that anti-GEP antibody treatment delayed tumor cell proliferation via the MAPK and AKT pathway in mouse tumor xenografts.

### Example 15

### Development of Anti-GEP Antibodies

GEP-specific antibodies were generated by immunizing BALB/c mice or New Zealand white rabbits with GEP specific peptide sequence located at and around SEQ ID No. 5, 6, 7, 8, 9, 10, 11, 12, or 13 (FIG. 11). The anti-GEP monoclonal antibodies or anti-GEP polyclonal antibodies were used to detect serum GEP levels or suppression of tumor growth.

### REFERENCES

1. El-Serag H B: Hepatocellular carcinoma: An epidemiologic view. J Clin Gastroenterol 2002; 35:S72-8.
2. Montalto G, Cervello M, Giannitrapani L, et al: Epidemiology, risk factors, and natural history of hepatocellular carcinoma. Ann N Y Acad Sci 2002; 963:13-20.
3. Befeler A S, Di Bisceglie A M: Hepatocellular carcinoma: diagnosis and treatment. Gastroenterology 2002; 122:1609-19.
4. Yu A S, Keeffe E B: Management of hepatocellular carcinoma. Rev Gastroenterol Disord 2003; 3:824.
5. Fan S T, Lo C M, Liu C L, et al: Hepatectomy for hepatocellular carcinoma: Toward zero hospital deaths. Ann Surg 1999; 229:322-30.
6. Ng K K, Lam C M, Poon R T et al: Thermal ablative therapy for malignant liver tumors: a critical appraisal. J Gastroenterol Hepatol 2003; 18:616-29.
7. MacDonald D J, Kelly A M: The rapid quantitation of serum alpha-fetoprotein by two-site micro enzyme immunoassay. Clin Chim Acta 1978:87:367-72.
8. Taketa K: Alpha-fetoprotein: reevaluation in hepatology. Hepatology 1990; 12:1420-32.
9. Johnson P J: The role of serum alpha-fetoprotein estimation in the diagnosis and management of hepatocellular carcinoma. Clin Liver Dis 2001:5:145-59.
10. Gebo K A, Chander G, Jenckes M W, et al: Screening tests for hepatocellular carcinoma in patients with chronic hepatitis C: a systematic review. Hepatology 2002; 36:S84-92.
11. Chen X, Cheung S T, So S, et al: Gene expression patterns in human liver cancers. Mol Biol Cell 2002; 13:1929-39.
12. Cheung S T, Wong S Y, Leung K L, et al: Granulin-epithelin precursor overexpression promotes growth and invasion of hepatocellular carcinoma. Clin Cancer Res 2004; 10:7629-36.
13. Guillemard V, Saragovi H U. Novel approaches for targeted cancer therapy. Curr Cancer Drug Targets 2004; 4:313-326.
14. Adams G P, Weiner L M. Monoclonal antibody therapy of cancer. Nat Biotechnol 2005; 23:1147-1157.
15. von Schilling C. Immunotherapy with anti-CD20 compounds. Semin Cancer Biol. 2003; 13:211-22.
16. Shak S. Overview of the trastuzumab (Herceptin) anti-HER2 monoclonal antibody clinical program in HER2-overexpressing metastatic breast cancer. Herceptin Multinational Investigator Study Group. Semin Oncol. 1999:71-7
17. Willems A, Gauger K, Henrichs C, Harbeck N. Antibody therapy for breast cancer. Anticancer Res. 2005; 25:1483-9.
18. Vanhoefer U, Tewes M, Rojo F, Dirsch O, Schleucher N, Rosen O, Tillner J, Kovar A, Braun A H, Trarbach T, Seeber S, Harstrick A, Baselga J. Phase I study of the humanized antiepidermal growth factor receptor monoclonal antibody EMD72000 in patients with advanced solid tumors that express the epidermal growth factor receptor. J Clin Oncol. 2004; 22:175-84.
19. Fernando N H, Hurwitz H I. Targeted therapy of colorectal cancer: clinical experience with bevacizumab. Oncologist. 2004; 9:11-8.
20. Thorgeirsson S S, Grisham J W: Molecular pathogenesis of human hepatocellular carcinoma. Nat Genet 2002; 31:339-46.
21. Kohler G, Milstein C: Continuous cultures of fused cells secreting antibody of predefined specificity. Nature 1975; 256:495-7.
22. Campbell A M: Production and purification of antibodies. In: Diamandis E P, Christopoulos T K, Eds. Immunoassay. Pp 95-15. San Diego, Calif.: Academic Press, 1996.
23. Zanocco-Marani T, Bateman A, Romano G, Valentinis B, He Z H, Baserga R. Biological activities and signaling pathways of the granulin/epithelin precursor. Cancer Res. 1999; 59:5331-40.
24. Shimada N, Ishii T, Imada T, Takaba K, Sasaki Y, Maruyama-Takahashi K, Maekawa-Tokuda Y, Kusaka H, Akinaga S, Tanaka A, Shitara K. A neutralizing anti-fibroblast growth factor 8 monoclonal antibody shows potent antitumor activity against androgen-dependent mouse mammary tumors in vivo. Clin Cancer Res 2005:11:3897-904.

TABLE 1
Diagnostic sensitivity of granulin-epithelin precursor (GEP) and α-
fetoprotein (AFP) in hepatocellular carcinoma patients (n = 107).

AFP

| GEP | <100 ng/ml | >100 ng/ml | Total |
|---|---|---|---|
| <9.07 ng/ml | 13 (12.1%) | 29 (27.1%) | 42 (39.3%) |
| >9.07 ng/ml | 32 (29.9%) | 33 (30.8%) | 65 (60.7%) |
| Total | 45 (42.1%) | 62 (58.0%) | 107 (100%) |

TABLE 2
Diagnostic sensitivity of granulin-epithelin precursor (GEP) and α-
fetoprotein (AFP) in different tumor stages of hepatocellular carcinoma
patients.

AFP

| | | | |
|---|---|---|---|
| GEP | <100 ng/ml | >100 ng/ml | Total |

Early Stage

| | | | |
|---|---|---|---|
| <9.07 ng/ml | 12 (15.8%) | 21 (27.6%) | 33 (43.4%) |
| >9.07 ng/ml | 22 (28.9%) | 21 (27.6%) | 43 (56.6%) |
| Total | 34 (44.7%) | 42 (55.3%) | 76 (100%) |

Late Stage

| | | | |
|---|---|---|---|
| <9.07 ng/ml | 1 (3.2%) | 8 (25.8%) | 9 (29.0%) |
| >9.07 ng/ml | 10 (32.3%) | 12 (38.7%) | 22 (71.0%) |
| Total | 11 (35.5%) | 20 (64.5%) | 31 (100%) |

Supplementary Table 1
Diagnostic sensitivity of granulin-epithelin precursor
(GEP) and α-fetoprotein (AFP) in hepatocellular carcinoma
patients (n = 107).

AFP

| GEP | <20 ng/ml | >20 ng/ml | Total |
|---|---|---|---|
| <9.07 ng/ml | 9 (8.4%) | 33 (30.8%) | 42 (39.3%) |
| >9.07 ng/ml | 24 (22.4%) | 41 (38.3%) | 65 (60.7%) |
| Total | 33 (30.8%) | 74 (69.2%) | 107 (100%) |

SUPPLEMENTARY TABLE 2
Diagnostic sensitivity of granulin-epithelin precursor
(GEP) and α-fetoprotein (AFP) in different tumor stages of hepatocellular
carcinoma patients.

AFP

| | | | |
|---|---|---|---|
| GEP | <20 ng/ml | >20 ng/ml | Total |

Early Stage

| | | | |
|---|---|---|---|
| <9.07 ng/ml | 9 (11.8%) | 24 (31.6%) | 33 (43.4%) |
| >9.07 ng/ml | 16 (21.1%) | 27 (35.5%) | 43 (56.6%) |
| Total | 25 (32.9%) | 51 (67.1%) | 76 (100%) |

Late Stage

| | | | |
|---|---|---|---|
| <9.07 ng/ml | 0 (0%) | 9 (29.0%) | 9 (29.0%) |
| >9.07 ng/ml | 8 (25.8%) | 14 (45.2%) | 22 (71.0%) |
| Total | 8 (25.8%) | 23 (74.2%) | 31 (100%) |

1. A method for detecting GEP protein in a biological sample, comprising the steps of:
incubating the sample in anti-GEP monoclonal-antibody-coated ELISA plates;
incubating the plates with anti-GEP polyclonal antibody; and
incubating the plates with horseradish peroxidase-conjugated anti-rabbit IgG;
incubating with TMB (3,3′,5,5′-tetramethylbenzidine); and
recording the optical density of the sample.

2. The method of claim 1, wherein the anti-GEP monoclonal antibody is generated in mice by GEP specific peptide.

3. The method of claim 1, wherein the anti-GEP monoclonal antibody is generated by GEP specific peptide as set forth in SEQ ID No. 3.

4. The method of claim 1, wherein the anti-GEP polyclonal antibody is generated by GEP specific peptide in rabbits.

5. The method of claim 1, wherein the anti-GEP polyclonal antibody is generated by GEP specific peptide as set forth in SEQ ID NO: 5.

6. A method for determining whether a subject has Hepatocellular carcinoma (HCC), comprising the steps of:
collecting a biological sample from the subject;
incubating the sample in anti-GEP monoclonal antibody coated ELISA plates;
incubating the plates with anti-GEP polyclonal antibody;
incubating the plates with horseradish peroxidase conjugated anti-rabbit IgG;
incubating the plates with TMB (3,3′,5,5′-tetramethylbenzidine);
recording the optical density of the sample,
determining the GEP level against a calibration curve of purified GEP; and
determining HCC risk in the sample by comparing GEP level against a known standard.

7. The method of claim 6, wherein the anti-GEP monoclonal antibody is generated in mice or rabbits by GEP specific peptide.

8. The method of claim 6, wherein the anti-GEP monoclonal antibody is generated by the GEP specific peptide forth in SEQ ID No. 3.

9. The method of claim 6, wherein the anti-GEP polyclonal antibody is generated by GEP specific peptide immunized in rabbits.

10. The method of claim 6, wherein the anti-GEP polyclonal antibody is generated by GEP specific peptide as set forth in SEQ ID NO: 5.

11. A method of suppressing hepatocellular carcinoma growth in a patient having hepatocellular carcinoma comprising administering to the patient an effective amount of anti-GEP antibody in a pharmaceutically effective vehicle.

12. The method of claim 11 wherein the anti-GEP antibody can be administered intraperitoneally, intravenously, or intratumorally.

13. The method of claim 1 wherein the anti-GEP monoclonal antibody is generated by GEP specific peptide in SEQ ID No. 2, located at or around regions as set forth in SEQ ID NOS: 5, 7, 9, 11, 13, 15, 17, 19, 21, or 23.

14. The method of claim 1 wherein the anti-GEP polyclonal antibody is generated by GEP specific peptide in SEQ ID No. 2, located at or around regions as set forth in SEQ ID NOS: 3, 7, 9, 11, 13, 15, 17, 19, 21, or 23.

15. The method of claim 6 wherein the anti-GEP monoclonal antibody is generated by GEP specific peptide in SEQ ID No. 2, located at or around regions as set forth in SEQ ID NOS: 5, 7, 9, 11, 13, 15, 17, 19, 21, or 23.

16. The method of claim 6 wherein the anti-GEP rabbit polyclonal antibody is generated by GEP specific peptide in SEQ ID No. 2, located at or around regions as set forth in SEQ ID NOS: 3, 7, 9, 11, 13, 15, 17, 19, 21, or 23.

17. A method of suppressing hepatocellular carcinoma (HCC) growth in a subject comprising administering to the subject an amount of anti-GEP monoclonal antibody as set forth in claim 15 effective to suppress HCC growth.

18. The method of claim 17 wherein the anti-GEP monoclonal antibody can be administered intraperitoneally, intravenously, or intratumorally.

19. A method of suppressing hepatocellular carcinoma growth in a subject with anti-GEP polyclonal antibody as set forth in claim 16.

20. The method of claim 19 wherein the anti-GEP polyclonal antibody can be administered intraperitoneally, intravenously, or intratumorally.

21. A pharmaceutical composition comprising an effective HCC cell proliferation or growth inhibiting amount of anti-GEP monoclonal antibody A23 in a pharmaceutically acceptable vehicle.

22. A method of suppressing HCC cell proliferation or growth in a mammal afflicted by HCC comprising administering to the mammal an amount of anti-GEP monoclonal antibody effective to suppress HCC cell proliferation or growth.

23. The method of claim 1, wherein the biological sample can be blood, serum, plasma, or urine.

24. The method of claim 6, wherein the biological sample can be blood, serum, plasma, or urine.

25. The method of claim 1, wherein the anti-GEP antibody is generated by reagents that involve the GEP specific region in SEQ ID No. 1, locates at or around the regions as set forth in SEQ ID NOS: 4, 6, 8, 10, 12, 14, 16, 18, 20, 22, or 24.

26. The method of claim 6, wherein the anti-GEP antibody is generated by reagents that involve the GEP specific region in SEQ ID No. 1, located at or around the regions as set forth in SEQ ID NOS: 4, 6, 8, 10, 12, 14, 16, 18, 20, 22, or 24.

27. The method of claim 11, wherein the anti-GEP antibody is generated by reagents that involve the specific region in SEQ ID No. 1, located at or around the regions as set forth in SEQ ID NOS: 4, 6, 8, 10, 12, 14, 16, 18, 20, 22, or 24.
